(19) United States
(12) Patent Application Publication
(10) Pub. No.: US 2017/0365148 A1
(43) Pub. Date: Dec. 21, 2017

(54) SYSTEM AND METHOD FOR FALL DETECTION

(71) Applicant: KONINKLIJKE PHILIPS N.V., EINDHOVEN (NL)

(72) Inventors: Brian Rosenfeld, Baltimore, MD (US); Steve Park, Baltimore, MD (US)

(21) Appl. No.: 15/534,745

(22) PCT Filed: Dec. 9, 2015
(86) PCT No.: PCT/IB2015/059476

§ 371 Date: Jun. 9, 2017

Related U.S. Application Data
(60) Provisional application No. 62/089,897, filed on Dec. 10, 2014.

(51) Int. Cl. G08B 21/04; A61B 5/11; A61B 5/00; A61B 5/0205; A61B 5/08; A61B 5/024; A61B 5/021

(52) U.S. Cl. CPC G08B 21/043; G08B 21/0446; A61B 5/746; A61B 5/1117; A61B 5/0205; A61B 5/1115; A61B 5/024; A61B 5/021; A61B 5/08

(57) ABSTRACT

The present system generates output signals conveying information related to a position of one or more body parts of the subject, location of the body of the subject and/or physiological information related to the subject; obtains a set of fall criteria that describe whether the subject is likely to fall; determines one or more body position parameters, one or more physiological parameters, and/or one or more body location parameters; compares the determined one or more physiological parameters, the one or more body position parameters, and/or the one or more body location parameters to criteria in the set of fall criteria; and, responsive to the one or more body position parameters, the one or more physiological parameters, and or the one or more body location parameters satisfying individual fall criteria in the set of fall criteria, generate an alert.

### BACKGROUND

### 1. Field

The present disclosure pertains to a system and method for fall detection for a subject.

### 2. Description of the Related Art

Known fall detection systems use simple pre-set bed boundaries and information from a single type of sensor to trigger an alarm when some part (e.g., even a finger) of the subject's body crosses a bed boundary. Currently, no systems exist that generate alerts in real time with a high degree of accuracy based on a determination that a subject has a high probability of falling out of their bed.

### SUMMARY

Accordingly, one or more aspects of the present disclosure relate to a system configured to generate a potential fall alert for a subject in a support structure. The system comprises one or more body position sensors, one or more physiological sensors, one or more physical computer processors, and/or other components. The one or more body position sensors may be configured to generate output signals conveying information related to a position of one or more body parts of the subject. The one or more physiological sensors may be configured to generate output signals conveying physiological information related to the subject. The one or more physical computer processors are configured by computer-readable instructions to obtain a set of fall criteria that describe whether the subject is likely to fall; determine one or more body position parameters, and one or more physiological parameters based on the output signals generated by the one or more body position sensors, and the one or more physiological sensors; compare the determined one or more physiological parameters, and the one or more body position parameters to criteria in the set of fall criteria, and responsive to the one or more body position parameters, and the one or more physiological parameters satisfying the criteria in the set of criteria, generate an alert.

Another aspect of the present disclosure relates to a method for generating a potential fall alert for a subject in a support structure with a system comprising one or more body position sensors, one or more physiological sensors, and one or more physical computer processors. The method comprises generating, with the one or more body position sensors, output signals conveying information related to a position of one or more body parts of the subject; generating, with the one or more physiological sensors, output signals conveying physiological information related to the subject; obtaining, with the one or more physical computer processors, a set of fall criteria that describe whether the subject is likely to fall; determining, with the one or more physical computer processors, one or more body position parameters, and one or more physiological parameters based on the output signals generated by the one or more body position sensors, and the one or more physiological sensors; comparing, with the one or more physical computer processors, the determined one or more physiological parameters, and the one or more body position parameters to criteria in the set of fall criteria; and responsive to the one or more body position parameters, and the one or more physiological parameters satisfying the criteria in the set of criteria, generating an alert with the one or more physical computer processors.

Still another aspect of the present disclosure relates to a system configured to generate a potential fall alert for a subject in a support structure. The system comprises means for generating output signals conveying information related to a position of one or more body parts of the subject; means for generating output signals conveying physiological information related to the subject; means for obtaining a set of fall criteria that describe whether the subject is likely to fall; means for determining one or more body position parameters, and one or more physiological parameters based on the output signals; means for comparing the determined one or more physiological parameters, and the one or more body position parameters to criteria in the set of fall criteria; and means for generating an alert responsive to the one or more body position parameters, and the one or more physiological parameters satisfying the criteria in the set of criteria.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured to generate a potential fall alert for a subject in a support structure.

FIG. 2 illustrates a subject in different body positions and body locations over time.

FIG. 3 illustrates a method for generating a potential fall alert for a subject in a support structure.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

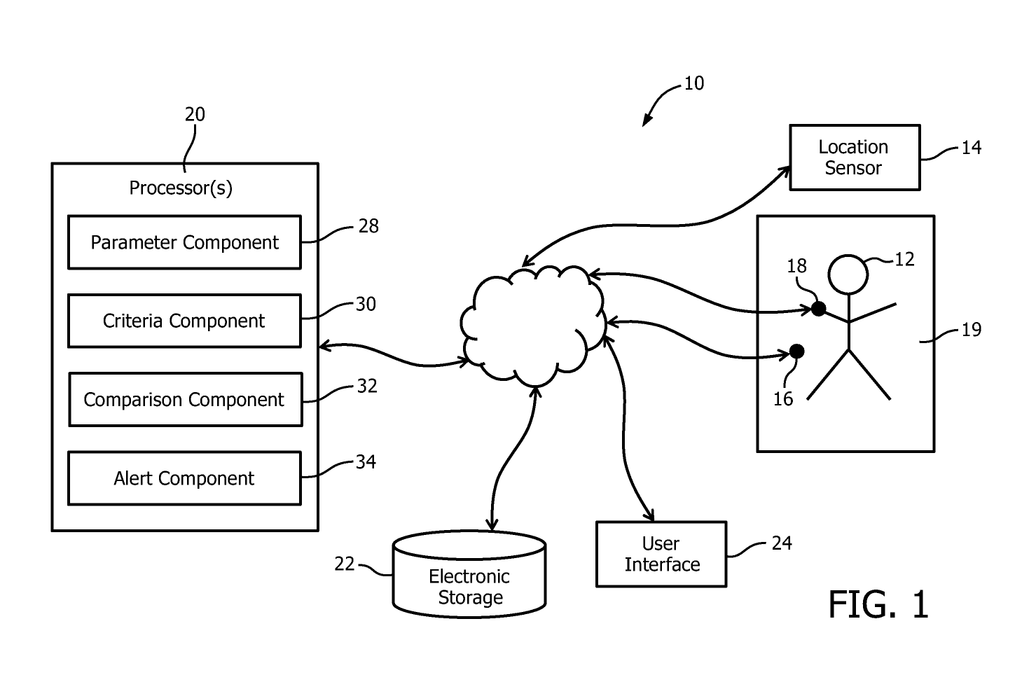

FIG. 1 is a schematic illustration of a system 10 configured to detect whether a subject 12 is at risk of falling from a support structure 19, in real or near real time, and to generate a potential fall alert. In some embodiments, support structure 19 may be one or more of a bed, a stretcher, a table, a chair, shoes, and/or other support structures. System 10 is configured to compare physiological, body location, and/or body position parameters of subject 12 to a set of fall criteria to determine whether subject 12 is likely to fall out of bed (for example). System 10 is configured to generate a potential fall alert responsive to one or more of the parameters satisfying one or more individual criteria in the criteria set. In some embodiments, system 10 comprises one or more of a body location sensor 14, a body position sensor 16, a physiological sensor 18, a support structure 19, a physical computer processor 20, electronic storage 22, a user interface 24, and/or other components. Components of system 10 may be configured to communicate via wire or wirelessly (e.g. via a wireless network).

In FIG. 1, body location sensors 14, body position sensors 16, physiological sensors 18, processor 20, electronic storage 22, and user interface 24 are shown as separate entities. This is not intended to be limiting. Some and/or all of the components of system 10 and/or other components may be grouped into one or more singular devices.

Body location sensor(s) 14 is configured to generate output signals conveying information related to location of the body of subject 12. In some embodiments, body location sensor(s) 14 may be configured to generate output signals conveying information related to location of the body of subject 12 relative to support structure 19, and/or other information.

In some embodiments, body location sensor(s) 14 include one or more of a video sensor, a motion sensor, a thermal sensor, an optical sensor, a photoelectric sensor, a PIR (pyroelectric infrared) sensor, a weight sensor, and or other sensors. By way of non-limiting example, body location sensor(s) 14 may be and/or include a video sensor. Although body location sensor(s) 14 is illustrated at a single location, this is not intended to be limiting. Body location sensor(s) 14 may include sensors disposed in a plurality of locations.

Body position sensor(s) 16 is configured to generate output signals conveying information related to a position of one or more body parts of subject 12. Body position may refer to the positions of body parts of subject 12 in relation to each other, orientation of one or more body parts, and/or other aspects of body position that are separate from location.

In some embodiments, body position sensor(s) 16 may include one or more of a motion sensor, a weight sensor, an optical sensor, a photoelectric sensor, a video sensor, a thermal sensor, and/or other sensors. For example, body position sensor(s) 16 may include a sensor worn on a specific body part of subject 12 configured to generate output signals conveying information about the physical location of that body part of subject 12. Although body position sensor(s) 16 is illustrated at a single location in communication with subject 12, this is not intended to be limiting. Body position sensor(s) 16 may include sensors disposed in a plurality of locations. It will be appreciated that an individual sensor may provide information relative to body location and body location. Such a sensor would be included in body location sensor(s) 14 and body position sensor(s) 16.

Physiological sensor(s) 18 is configured to generate output signals conveying physiological information related to the subject. In some embodiments, physiological sensors may include a pulse oximeter configured to monitor the oxygen saturation of a subject's blood, a cardiac monitor to monitor, for example, subject 12's cardiac rhythm and/or heart rate variability, a breathing sensor configured to monitor the respiration and/or respiration rate of subject 12, and/or other physiological sensors.

In some embodiments, physiological sensor(s) may generate output signals which convey information related to the subject heart rate, blood pressure, respiration, pulse rate, respiratory effort, blood oxygenation, and/or other physiological information. Although physiological sensor(s) 18 is illustrated at a single location in communication with subject 12, this is not intended to be limiting. Physiological sensor(s) 18 may include sensors disposed in a plurality of locations.

In some embodiments, the output signals generated by the body location sensor(s) 14, the body position sensor(s) 16, the physiological sensor(s) 18, and/or other sensors may include one or more of a video stream, an audio stream, a visual indicator, a numerical data stream, and/or other forms of information.

Processor(s) 20 is configured to provide information processing capabilities in system 10. As such, processor 20 may comprise one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may comprise a plurality of processing units. These processing units may be physically located within the system 10, or processor(s) 20 may represent processing functionality of a plurality of devices operating in coordination.

As shown in FIG. 1, processor 20 is configured to execute one or more computer program components. The one or more computer program components may comprise one or more of a parameter component 28, a criteria component 30, a comparison component 32, an alert component 34, and/or other components. Processor(s) 20 may be configured to execute components 28, 30, 32, and/or 34 by software; hardware; firmware; some combination of software, hardware, and/or firmware; and/or other mechanisms for configuring processing capabilities on processor(s) 20.

It should be appreciated that although components 28, 30, 32, and 34 are illustrated in FIG. 1 as being co-located within a single processing unit, in embodiments in which processor(s) 20 comprises multiple processing units, one or more of components 28, 30, 32, and/or 34 may be located remotely from the other components. The description of the functionality provided by the different components 28, 30, 32, and/or 34 described below is for illustrative purposes, and is not intended to be limiting, as any of components 28, 30, 32, and/or 34 may provide more or less functionality than is described. For example, one or more of components 28, 30, 32, and/or 34 may be eliminated, and some or all of its functionality may be provided by other components 28, 30, 32, and/or 34. As another example, processor 20 may be configured to execute one or more additional components that may perform some or all of the functionality attributed below to one of components 28, 30, 32, and/or 34.

Parameter component 28 is configured to determine one or more parameters related to subject 12. The one or more parameters include one or more body location parameters, one or more body position parameters, one or more physiological parameters, and/or other parameters. In some embodiments, parameter component 28 may be configured to determine the one or more parameters based on the output signals generated by body location sensors 14, body position sensors 16, physiological sensors 18, and/or other sensors. In some embodiments, parameter component 28 may be configured to obtain the one or more parameters related to subject 12 based on a user input (e.g. input from a user via user interface 24), and/or based on information received by other mechanisms. In some embodiments, parameter component 28 is configured to determine the one or more parameters in an ongoing manner over time. In some embodiments, parameter component 28 is configured to determine the one or more parameters at regular intervals of time and/or at other times. The regular intervals of time may be pre-determined at manufacture, set by a user via user interface 24, and/or determined in other ways.

In some embodiments, the one or more body location parameters may include parameters related to location of the body of subject 12. In some embodiments, the body location parameters may be related to and/or indicate location of the body of subject 12 relative to some reference frame (e.g., a room, a support structure 19, and/or other reference frames). In some embodiments, the one or more body location parameters may indicate location of a specific body part or set of body parts (e.g., an arm, the location of a leg, the location of a head, the location of a torso). The one or more body location parameters may specify or characterize the location of the body as a whole (e.g., a center of mass, a silhouette, a furthest coordinate location of any body part in two or more different directions, and/or other techniques to characterize location of the body as a whole).

The one or more body position parameters may be related to a body position of subject 12. Body position may refer to the positions of body parts of subject 12 in relation to each other, orientation of one or more body parts, and/or other aspects of body position that are separate from location. Parameters related to a position of one or more body parts of subject 12 may include parameters related to a position of one or more body parts relative to other body parts (e.g., a distance, angle, etc. between body parts that indicate things like outstretched arms, tilted head/neck, etc.), parameters related to a collective position of body parts that indicates one or more postures of subject 12 (e.g., standing, seated, lying down, rolled on his/her side, lying in a fetal position, etc.), parameters related to an orientation of one or more body parts (e.g., facing a specific direction, laying on his/her stomach, laying on his/her back), and/or other parameters. Examples of a body position of subject 12 in support structure 19 may be and/or include one or more of Fowler's position, a supine position, a lateral recumbent position, a prone position, and/or other body positions.

By way of non-limiting example, body position sensor(s) 16, described above, may include a weight distribution sensor configured to generate output signals conveying information about the body position (weight distribution) of the body of subject 12. In this example, the information conveyed by the output signals indicates the body position of subject 12 by way of the weight distribution on support structure 19. Continuing with the example, the output signals may indicate that subject 12 is in an upright position based on his weight distribution on support structure 19. In this example a change in the body position may be a potential fall indicative.

In some embodiments, the one or more physiological parameters include cardiac parameters, respiratory parameters, and/or other parameters. In some embodiments, the cardiac activity parameters include parameters related to and/or determined based on an electrocardiogram of subject 12, and/or other parameters. In some embodiments, the respiratory parameters may include parameters indicative of respiratory effort in subject 12, a respiration rate of subject 12, a flow rate (e.g., of gas into and out of subject 12 during respiration), a volume (e.g., a tidal volume of inhaled and/or exhaled gas), a pressure (e.g., an inhalation pressure, an exhalation pressure), an amplitude (e.g., of pressure and/or any other parameter of inhaled and/or exhaled gas), and/or other parameters. In some embodiments, the respiration parameters may include a parameter indicative of variation in the respiration rate of subject 12 (and/or variation of any other parameter).

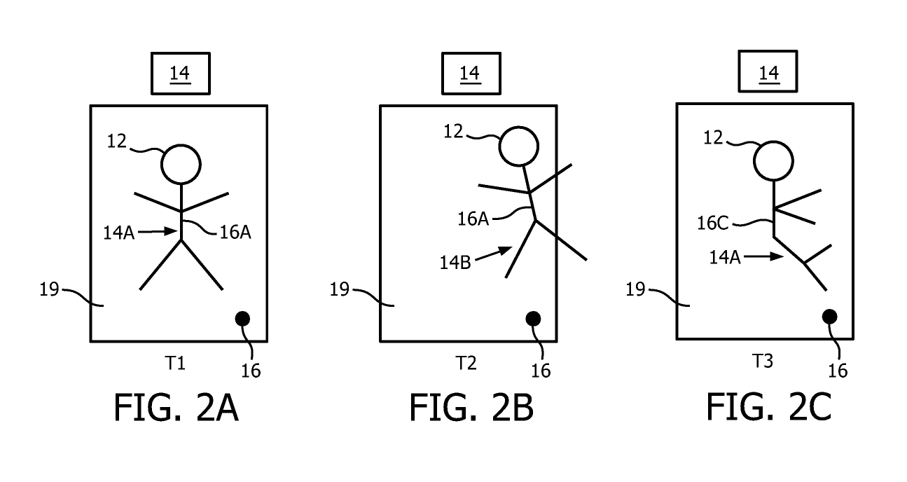

By way of non-limiting example, FIG. 2 illustrates subject 12 on a support structure 19. Location sensor(s) 14 and body position sensor(s) 16 generate output signals conveying information regarding subject 12's body position and body location over time. FIG. 2 A shows an example of subject 12, at time T1, on support structure 19 in a body position 16 A and in a body location 14 A. FIG. 2 B shows subject 12, at time T2, on support structure 19 in same body position 16A, but in a different body location 14B. FIG. 2C shows subject 12, at time T3, on support 19 in a different body position 16C.

In some embodiments, parameter component 28 is configured to obtain initial baseline parameters related to subject 12. Initial baseline parameters may be obtained and/or determined based on the output signals of sensor(s) 14, sensor(s) 16, sensor(s) 18; from information entered and/or received via user interface 24, based on information determined by processor(s) 20, based on information stored in electronic storage 22, based on information communicated via a network, and/or other information. Initial baseline parameters may be initial values of one or more parameters at the beginning of subject 12's monitoring, for example. Initial baseline parameters may include initial baseline physiological parameters, initial baseline body position parameters, initial baseline body location parameters, and/or other initial baseline parameters related to subject 12. For example, initial baseline physiological parameters, obtained at the beginning of subject 12's monitoring, may include subject 12's initial heart rate, initial blood pressure, initial respiratory rates, and/or other initial physiological parameters.

As a further example, initial baseline body position parameters may include an initial body position of subject 12 in support structure 19. For example at the beginning of the monitoring of subject 12, the body of subject 12 is initially positioned on support structure 19 in a specific position (e.g. supine). In this example, the supine position would be the initial baseline body position parameter.

As a further example, initial baseline body location parameters may include an initial body location of one or more body parts of subject 12 relative to support structure 19. For example at the beginning of the monitoring of subject 12, the body of subject 12, and/or all other body parts of subject 12 may be inside and/or otherwise within support structure 19. In this example, the locations of body parts of subject 12 contained within support structure 19 would the initial baseline body location parameters.

Criteria component 30 is configured to obtain a set of fall criteria that describe whether subject 12 is likely to fall. The set of fall criteria may be comprised of one or more individual criteria. In some embodiments, criteria component 30 is configured to obtain one or more sets of fall criteria such that individual sets of fall criteria may be comprised of one or more sets of fall criteria. Individual sets of fall criteria may include different quantities of individual criteria (e.g., a first set of fall criteria may include one individual criterion and a second set of fall criteria may include ten individual criteria). The individual criteria in a criteria set may correspond to one or more parameters determined by the parameters component. In some embodiments, an individual criteria set includes at least one individual criterion that corresponds to a physiological parameter. In some embodiments, an individual criteria set includes at least one individual criterion that corresponds to a physiological parameter and at least one individual criterion that corresponds to a body position parameter. In some embodiments, an individual criteria set includes at least one individual criterion that corresponds to a physiological parameter, at least one individual criterion that corresponds to a body position parameter, and at least one individual criterion that corresponds to a body location parameter.

By way of non-limiting example, a set of fall criteria may include a predetermined heart rate criteria threshold value, a predetermined respiration rate criteria threshold value, a combination heart rate/respiration rate criterion, a range of (e.g., high risk as described below) body locations, and one or more (e.g., high risk as described below) body positions. In this example, if the criteria in the set are satisfied at any given time, then the subject is at high risk of falling.

In some embodiments, the sets of fall criteria include sets of fall criteria determined by a user (e.g. healthcare professional, subject caregiver, etc.), by a manufacturer, criteria determined based on policies and/or procedures associated with a facility (e.g. hospital, clinic, rehabilitation facility, nursing home, etc.), criteria determined by subject 12 (e.g., via user interface 24), criteria determined by processor(s) 20, and/or other criteria. In some embodiments, the sets of fall criteria include sets of fall criteria specific to subject 12. For example, sets of fall criteria specific to subject 12 may be determined based on one or more of the age, gender, height, weight, heart rate, blood pressure, medical condition, physical condition, medication history, psychological condition, and/or other information specific to subject 12. In some embodiments, the sets of fall criteria include sets of fall criteria that are the same for all subjects. For example, one criterion that is the same for all subjects may be a heart rate equal to or higher than a predetermined value regardless of the subject. Another example of a criterion that is the same for all subjects may be a body part location in a predetermined area (e.g. head of subject is off the support structure) regardless of the identity of subject being monitored.

In some embodiments, criteria component 30 is configured such that the sets of fall criteria are determined based on the initial baseline parameters specific to subject 12. For example, the sets of fall criteria may be determined based on the initial baseline physiological, body position, body location, and/or other initial baseline parameters specific to subject 12 obtained by parameter component 28 at the beginning of the monitoring of subject 12.

In some embodiments, criteria component 30 is configured to adjust the sets of fall criteria based on the baseline parameters, changes in the determined parameters, and/or other information. For example, at the beginning of monitoring of subject 12, who is heavily sedated, the initial baseline parameters specific to subject 12 are obtained by parameter component 28. In this example, criteria component 30 is configured such that the initial baseline parameters are used to determine one or more of the sets of fall criteria. As the sedation fades away, parameter component 28 may be configured to determine new non-sedated baseline parameters and one or more of the criteria sets are adjusted (e.g. by a user, by criteria component 30, and/or other mechanisms).

In some embodiments, one or more of the criteria sets may be associated with one or more falling risk levels. For example, in some embodiments, criteria component 30 is configured such that one or more criteria sets are associated with a low risk of falling, one or more criteria sets are associated with a high risk of falling, and/or one or more criteria sets are associated with a panic risk of falling. In some embodiments, criteria component 30 is configured such that one or more of the criteria sets may include one or more individual criteria that are weighted more heavily or lightly than other individual criteria. For example, body position and/or body location criteria that indicate (when satisfied) that one or more portions of subject 12's body is hanging over an edge of support structure 19 may be more heavily weighted than criteria indicating (when satisfied) that the heart rate of subject 12 has increased. Criteria indicating (when satisfied) that the heart rate of subject 12 has jumped significantly may be more heavily weighted than criteria indicating (when satisfied) that the heart rate of subject 12 has increased only a small amount.

Comparison component 32 is configured to compare the determined parameters to the set of fall criteria. In some embodiments, the one or more physiological parameters, the one or more body position parameters, the one or more body location parameters, and/or other parameters are compared to criteria in the set of fall criteria. In some embodiments, comparison component 32 is configured to compare the determined parameters to one or more sets of fall criteria. In some embodiments, comparison component 32 compares one or more parameters to a first set of fall criteria. For example, the one or more parameters may include any combination of one or more of physiological parameters, body position parameters, body location parameters, and/or other parameters. In some embodiments, comparison component 32 compares the determined one or more physiological parameters, one or more body position parameters, and/or one or more body location parameters to the initial baseline parameters and then to the fall criteria. In some embodiments, comparison component 32 is configured to compare the physiological parameters over time, the speed at which the physiological parameters change over time, and/or other information to corresponding fall criteria. In some embodiments, comparison component 32 is configured to compare the body position parameters over time, the speed at which the body position parameters change over time, and/or other information to corresponding fall criteria. In some embodiments, comparison component 32 is configured to compare the body location parameters over time, the speed at which the body location parameters change over time, and/or other information to corresponding fall criteria.

Alert component 34, is configured to generate an alert responsive to the one or more body position parameters, the one or more physiological parameters, and/or the one or more body location parameters satisfying the set of fall criteria. In some embodiments, alert component 34 is configured such that the alert generated is one or more of a sound alert, a visual alert, an email alert, a text alert, a message alert, a vibration alert, and/or other types of alerts. In some embodiments, the alert generated by the alert component 34 may be generated via speakers, video monitors, user interface 24, phones systems, marquee signs, cell phones, computers, other components of system 10, and/or other mediums for generating an alert.

In some embodiments, alert component 34, may be configured to generate individual types of alerts that correspond to satisfaction of individual sets of fall criteria (e.g., panic risk criteria sets, high risk criteria sets, low risk criteria sets). For example, alert types may include a mild alert, a high alert, a panic alert, a status alert, and/or other types of alerts. A mild alert may be generated when the risk of the subject falling is low (e.g., responsive to criteria in a low risk criteria set being satisfied), a high alert may be generated when the risk of the subject falling is high (e.g., responsive to criteria in a high risk criteria set being satisfied), and a panic alert may be generated when the risk of the subject falling is imminent (e.g., responsive to criteria in a panic risk criteria set being satisfied). A status alert may be generated to send a status update of subject. For example, alert component 34, may send a status update, at predetermined intervals of time (e.g. every 60 seconds), relaying the risk of falling for subject 12 at the current time. Example of status update may include emitting a green light when subject 12 is not at risk of falling. A further example of a status update may be emitting a beep/or sound when subject 12 is not at risk of falling, and/or other methods for generating a status update. In some embodiments, a high and/or panic risk alert may be generated responsive to heavily weighted criteria in one or more criteria sets being satisfied.

In some embodiments, alert component 34, is configured to generate an alert responsive to the one or more body position parameters, the one or more physiological parameters, and/or the one or more body location parameters satisfying one or more sets of fall criteria.

Electronic storage 22 comprises electronic storage media that electronically stores information. The electronic storage media of electronic storage 22 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 22 may store software algorithms, information determined by processor 20, information received from subject 12, and/or other information that enables system 10 to function properly. Electronic storage 22 may be (in whole or in part) a separate component within system 10, or electronic storage 22 may be provided (in whole or in part) integrally with one or more other components of system 10 (e.g., processor 20).

User interface 24 is configured to provide an interface between system 10 and subject 12, and/or other users through which subject 12 and/or the other users may provide information to and receive information from system 10. Other users may include doctors, caregivers, and/or other users, for example. This enables data, cues, results, and/or instructions and any other communicable items, collectively referred to as “information,” (e.g., the alerts) to be communicated between a user (e.g., subject 12) and one or more of body location sensor(s) 14, body position sensor(s), physiological sensor(s) 18, processor(s) 20, electronic storage 22, user interface 24, and/or other components of system 10.

Examples of interface devices suitable for inclusion in user interface 24 comprise a keypad, buttons, switches, a keyboard, knobs, levers, a display screen, a touch screen, speakers, a microphone, an indicator light, an audible alarm, a printer, a tactile feedback device, and/or other interface devices. In some embodiments, user interface 24 comprises a plurality of separate interfaces. In some embodiments, user interface 24 comprises at least one interface that is provided at or near support structure 19 and at least one interface located remotely (e.g., at a nurse's station in a care facility) from support structure 19.

It is to be understood that other communication techniques, either hard-wired or wireless, are also contemplated by the present disclosure as user interface 24. For example, the present disclosure contemplates that user interface 24 may be integrated with a removable storage interface provided by electronic storage 22. In this example, information may be loaded into system 10 from removable storage (e.g., a smart card, a flash drive, a removable disk, etc.) that enables the user(s) to customize the implementation of system 10. Other exemplary input devices and techniques adapted for use with system 10 as user interface 24 comprise, but are not limited to, an RS-232 port, RF link, an IR link, modem (telephone, cable or other). In short, any technique for communicating information with system 10 is contemplated by the present disclosure as user interface 24.

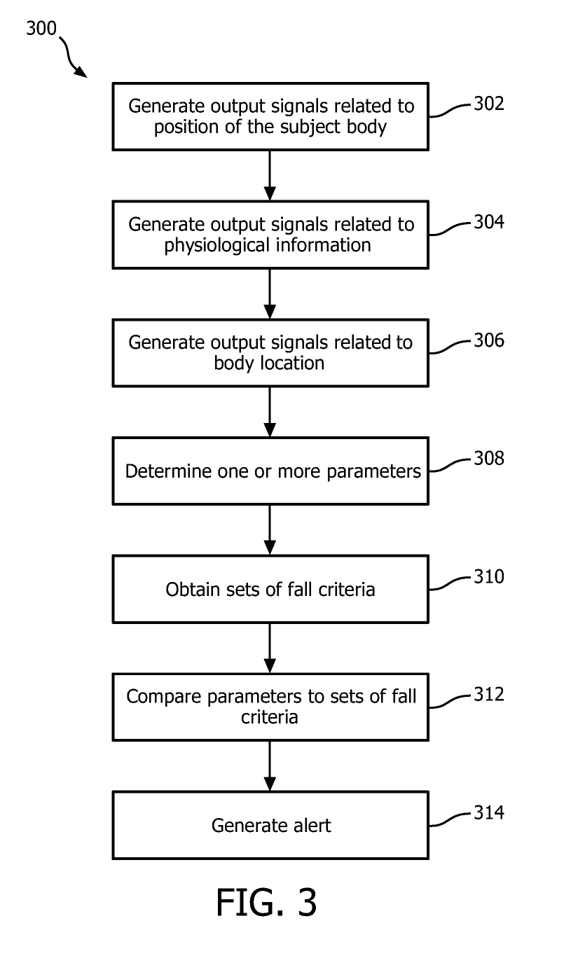

FIG. 3 illustrates a method 300 for generating a potential fall alert for a subject in a support structure with a potential fall alert generating system. The potential fall alert generating system comprises one or more body position sensors, one or more physiological sensors, one or more body location sensors, one or more physical computer processors, and/or other components. The operations of method 300 presented below are intended to be illustrative. In some embodiments, method 300 may be accomplished with one or more additional operations not described, and/or without one or more of the operations discussed. Additionally, the order in which the operations of method 300 are illustrated in FIG. 3 and described below is not intended to be limiting.

In some embodiments, method 300 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 300 in response to instructions stored electronically on an electronic storage medium. The one or more processing devices may include one or more devices configured through hardware, firmware, and/or software to be specifically designed for execution of one or more of the operations of method 300.

At an operation 302, output signals conveying information related to a position of one or more body parts of the subject are generated. In some embodiments, operation 302 is performed by one or more body position sensors the same as or similar to sensors 16 (shown in FIG. 1 and described herein).

At an operation 304, output signals conveying physiological information related to the subject are generated. In some embodiments, operation 304 is performed by one or more physiological sensors the same as or similar to sensors 18 (shown in FIG. 1 and described herein).

At an operation 306, output signals conveying information related to location of the body of the subject are generated. In some embodiments, operation 306 is performed by one or more body location sensors the same as or similar to sensors 14 (shown in FIG. 1 and described herein).

In some embodiments, the one or more body position sensors, the one or more physiological sensors, and/or the one or more body location sensors, may include one or more of a motion sensor, a position sensor, a weight sensor, a video sensor, a thermal sensor, an optical sensor, a photoelectric sensor, a voice sensor, a sound sensor, a heart rate sensor, a blood pressure sensor, a respiration sensor, a pulse sensor, and/or other types of sensors.

At an operation 308, one or more body position parameters, one or more physiological parameters, one or more body location parameters, and/or other parameters are determined. In some embodiments, the one or more body position parameters, the one or more physiological parameters, and/or the one or more body location parameters are determined based on the output signals generated by the one or more body position sensors, the one or more physiological sensors, and/or the one or more body location sensors. In some embodiments, operation 308 is performed by a physical computer processor the same as or similar to processor 20 (shown in FIG. 1 and described herein).

At an operation 310, a set of fall criteria that describe whether the subject is likely to fall, is obtained. In some embodiments, operation 310 comprises obtaining initial baseline parameters related to the subject. In some embodiments, the initial baseline parameters include baseline physiological parameters, baseline body position parameters, baseline body location parameters, and/or other baseline parameters. In some embodiments, the set of fall criteria may be determined, and/or adjusted based on one or more of the output signals, based on the initial baseline parameters, and/or based on other parameters. In some embodiments, the set of fall criteria may be determined by a user, a processor (e.g., such as processor 20), other components of system 10, and/or other fall criteria determination mechanisms. In some embodiments, operation 310 comprises obtaining one or more sets of fall criteria such that the sets of fall criteria include a first set of fall criteria. In some embodiments, operation 310 is performed by a physical computer processor the same as or similar to processor 20 (shown in FIG. 1 and described herein).

At an operation 312, the one or more determined physiological parameters, the one or more determined body position parameters, and/or the one or more determined body location parameters are compared to criteria in the set of fall criteria. In some embodiments, one or more physiological parameters, one or more body position parameters, and/or one or more body location parameters are compared to individual criteria in the set of fall criteria. In some embodiments, operation 312 includes comparing the initial baseline parameters to the one or more physiological parameters, the one or more body position parameters, and/or the one or more body location parameters. In some embodiments, operation 312 is performed by a physical computer processor the same as or similar to processor 20 (shown in FIG. 1 and described herein).

At an operation 314, an alert is generated responsive to the one or more body position parameters, the one or more physiological parameters and/or the one or more body location parameters satisfying the set of fall criteria. In some embodiments, an alert is generated responsive to the one or more body position parameters, the one or more physiological parameters and/or the one or more body location parameters satisfying one or more sets of fall criteria such that the one or more sets of fall criteria includes a first set of fall criteria. In some embodiments, an alert is generated responsive to one or more of a first body position parameter, a first physiological parameter, and/or a first body location parameter satisfying the first set of fall criteria. In some embodiments, the alert generated is one or more of a sound alert, a visual alert, an email alert, a text alert, a vibration alert, and/or other types of alerts. In some embodiments, the type and/or level of an alert generated may correspond to individual sets of fall criteria. For example, a mild alert may be generated when the risk of the subject falling is mild (e.g., criteria indicating that the risk of the subject falling from bed is mild are satisfied by the parameters). A high alert may be generated when the risk of the subject falling is high (e.g., criteria indicating that the risk of the subject falling from bed is high are satisfied by the parameters). A panic alert may be generated when the risk of the subject falling is imminent (e.g., criteria indicating that the subject is falling and/or about to fall from bed is satisfied by the parameters). In some embodiments, a status alert may be generated to send a status of a subject regardless whether the subject is at risk of falling. In some embodiments, operation 214 is performed by a physical computer processor the same as or similar to processor 20 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system that generates a potential fall alert for a subject in a support structure, the system comprising:
one or more body position sensors that generate output signals conveying information related to a position of one or more body parts of the subject;
one or more physiological sensors that generate output signals conveying physiological information related to the subject; and
one or more physical computer processors comprising computer-readable instructions to:
obtain a set of fall criteria that describe whether the subject is likely to fall;
determine one or more body position parameters, and one or more physiological parameters based on the output signals generated by the one or more body position sensors, and the one or more physiological sensors;
compare the determined one or more physiological parameters, and the one or more body position parameters to criteria in the set of fall criteria; and
responsive to the one or more body position parameters, and the one or more physiological parameters satisfying the fall criteria in the set of fall criteria, generate an alert.

2. The system of claim 1, wherein the one or more body position sensors, and/or the one or more physiological sensors comprise at least one of: a motion sensor, a position sensor, a weight sensor, an optical sensor, a photoelectric sensor, a voice sensor, a sound sensor, a heart rate sensor, a blood pressure sensor, a respiration sensor, and a pulse sensor.

3. The system of claim 1, wherein the one or more physical computer processors are further configured to obtain initial baseline parameters related to the subject, the initial baseline parameters including baseline physiological parameters and baseline body position parameters, and
wherein comparing the determined one or more physiological parameters, and the one or more body position parameters to criteria in the set of fall criteria includes comparing the initial baseline parameters to the determined one or more physiological parameters, and the determined one or more body position parameters.

4. The system of claim 3, wherein the one or more physical computer processors are configured to determine the set of fall criteria based on the output signals and/or the initial baseline parameters.

5. The system of claim 1, further comprising one or more body location sensors that generate output signals conveying information related to location of the body of the subject, and wherein the one or more physical computer processors further:
determine the one or more body position parameters, the one or more physiological parameters, and one or more body location parameters based on the output signals generated by the one or more body position sensors, the one or more physiological sensors, and the one or more body location sensors;
compare the determined one or more physiological parameters, the one or more body position parameters, and the one or more body location parameters to criteria in the set of fall criteria, and
responsive to the one or more body position parameters, the one or more physiological parameters, and the one or more body location parameters satisfying the fall criteria in the set of fall criteria, generate an alert.

6. A method for generating a potential fall alert for a subject in a support structure with a system comprising one or more body position sensors, one or more physiological sensors, and one or more physical computer processors, the method comprising:
generating, with the one or more body position sensors, output signals conveying information related to a position of one or more body parts of the subject;
generating, with the one or more physiological sensors, output signals conveying physiological information related to the subject;
obtaining, with the one or more physical computer processors, a set of fall criteria that describe whether the subject is likely to fall; determining, with the one or more physical computer processors, one or more body position parameters, and one or more physiological parameters based on the output signals generated by the one or more body position sensors, and the one or more physiological sensors;
comparing, with the one or more physical computer processors, the determined one or more physiological parameters, and the one or more body position parameters to criteria in the set of fall criteria; and
responsive to the one or more body position parameters, and the one or more physiological parameters satisfying the fall criteria in the set of fall criteria, generating an alert with the one or more physical computer processors.

7. The method of claim 6, wherein the one or more body position sensors, and/or the one or more physiological sensors comprise at least one of: a motion sensor, a position sensor, a weight sensor, an optical sensor, a photoelectric sensor, a voice sensor, a sound sensor, a heart rate sensor, a blood pressure sensor, a respiration sensor, or a pulse sensor.

8. The method of claim 6, further comprising obtaining, with the one or more physical computer processors, initial baseline parameters related to the subject, the initial baseline parameters including baseline physiological parameters and baseline body position parameters, and
wherein comparing the determined one or more physiological parameters, and the one or more body position parameters to criteria in the set of fall criteria includes comparing the initial baseline parameters to the determined one or more physiological parameters, and the determined one or more body position parameters.

9. The method of claim 8, further comprising determining, with the one or more physical computer processors, the set of fall criteria based on the output signals and/or the initial baseline parameters.

10. The method of claim 6, further comprising generating, with one or more body location sensors, output signals conveying information related to location of the body of the subject, and
determining, with the one or more physical computer processors, the one or more body position parameters, the one or more physiological parameters, and one or more body location parameters based on the output signals generated by the one or more body position sensors, the one or more physiological sensors, and the one or more body location sensors;
comparing, with the one or more physical computer processors, the determined one or more physiological parameters, the one or more body position parameters, and the one or more body location parameters to criteria in the set of fall criteria, and
responsive to the one or more body position parameters, the one or more physiological parameters, and the one or more body location parameters satisfying the fall criteria in the set of fall criteria, generating an alert with the one or more physical computer processors.

11. A system to generate a potential fall alert for a subject in a support structure, the system comprising:
means for generating output signals conveying information related to a position of one or more body parts of the subject;
means for generating output signals conveying physiological information related to the subject;
means for obtaining a set of fall criteria that describe whether the subject is likely to fall;
means for determining one or more body position parameters, and one or more physiological parameters based on the output signals;
means for comparing the determined one or more physiological parameters, and the one or more body position parameters to criteria in the set of fall criteria; and
means for generating an alert responsive to the one or more body position parameters, and the one or more physiological parameters satisfying the fall criteria in the set of fall criteria.

12. The system of claim 11, wherein the means for generating output signals conveying information related to a position of one or more body parts of the subject, and the means for generating output signals conveying physiological information related to the subject comprises at least one of, a motion sensor, a position sensor, a weight sensor, an optical sensor, a photoelectric sensor, a voice sensor, a sound sensor, a heart rate sensor, a blood pressure sensor, a respiration sensor, or a pulse sensor.

13. The system of claim 11, further comprising means for obtaining initial baseline parameters related to the subject, the initial baseline parameters including baseline physiological parameters and baseline body position parameters, and
wherein comparing the determined one or more physiological parameters, and the one or more body position parameters to fall criteria in the set of fall criteria includes comparing the initial baseline parameters to the determined one or more physiological parameters, and the determined one or more body position parameters.

14. The system of claim 13, further comprising means for determining the set of fall criteria based on the output signals and/or the initial baseline parameters.

15. The system of claim 11, further comprising
means for generating output signals conveying information related to location of the body of the subject,
means for determining the one or more body position parameters, the one or more physiological parameters, and one or more body location parameters based on the output signals;
means for comparing the determined one or more physiological parameters, the one or more body position parameters, and the one or more body location parameters to fall criteria in the set of fall criteria, and
means for generating an alert responsive to the one or more body position parameters, the one or more physiological parameters, and the one or more body location parameters satisfying the fall criteria in the set of fall criteria.
